(19) United States
(12) Patent Application Publication
(10) Pub. No.: US 2011/0118549 A1
(43) Pub. Date: May 19, 2011

(54) Endoscopic camera module package and method of manufacturing the same

(75) Inventor: Joon Hyuk Han, Hwaseong (KR)

(73) Assignee: SAMSUNG ELECTRO-MECHANICS CO., LTD., Suwon (KR)

(21) Appl. No.: 12/926,081

(22) Filed: Oct. 25, 2010

(30) Foreign Application Priority Data
Nov. 16, 2009 (KR) 10-2009-0110335

(51) Int. Cl. A61B 1/04; H01R 43/00

(52) U.S. Cl. 600/109; 29/854

(57) ABSTRACT

There is provided an endoscopic camera module package including: a lens part including at least one lens arranged along an optical axis; a flexible printed circuit board having an image sensor mounted thereon, the image sensor allowing light passing through the lens part to form an image; and a housing including an image sensor supporting frame allowing the image sensor to be supported at a rear of the lens part while the flexible printed circuit board is bent.

### CROSS-REFERENCE TO RELATED APPLICATIONS

This application claims the priority of Korean Patent Application No. 10-2009-0110335 filed on Nov. 16, 2009, in the Korean Intellectual Property Office, the disclosure of which is incorporated herein by reference.

### BACKGROUND OF THE INVENTION

1. Field of the Invention

The present invention relates to an endoscopic camera module package, and more particularly, to an endoscopic camera module package allowing for sufficient lighting and an increase in productivity due to a simplified manufacturing process, and a method of manufacturing the endoscopic camera module package.

2. Description of the Related Art

In general, an endoscope is an instrument used for visual inspection and diagnosis of an abnormality on the internal parts of a human body.

Besides the endoscope, an X-ray fluoroscope or an ultrasonic probe may be used as an instrument for observing images of the internal parts of a human body. However, the endoscope is distinguished from those devices in that it is directly inserted into the human body in order to view an abnormality.

Since the endoscope is put into the human body so that the parts inside can be seen, sufficient lighting must be ensured in order to capture clear images.

Therefore, in order to ensure sufficient lighting by illuminating a subject without the blocking of a lens part, a light source part of an endoscopic camera is usually positioned at substantially the same height as the front surface of the lens part.

In order to position the light source part at substantially the same height as the front surface of the lens part, a separate printed circuit board (PCB) having the light source part mounted thereon is prepared, and then this PCB is connected to a PCB having an image sensor mounted thereon or is dealt with a separate wiring. However, those methods have a problem of an addition in the number of required elements and processes.

### SUMMARY OF THE INVENTION

An aspect of the present invention provides an endoscopic camera module package allowing for sufficient lighting by arranging a light source part closer to the front surface of a lens part, as well as an increase in productivity due to a simplified manufacturing process.

According to an aspect of the present invention, there is provided an endoscopic camera module package including: a lens part including at least one lens arranged along an optical axis; a flexible printed circuit board having an image sensor mounted thereon, the image sensor allowing light passing through the lens part to form an image; and a housing including an image sensor supporting frame allowing the image sensor to be supported at a rear of the lens part while the flexible printed circuit board is bent.

The image sensor supporting frame may allow the flexible printed circuit board to be bent by being formed to be spaced apart from the lens part at the rear of the lens part while having a predetermined height in a direction perpendicular to the optical axis.

The flexible printed circuit board may have a light source part mounted thereon in order to provide lighting for photography, and the light source part may be positioned in front of the image sensor.

The light source part may be a chip light emitting diode (LED).

The housing may include a light source coupling part allowing the light source part to be coupled with the housing in a direction perpendicular to the optical axis.

The housing may include a lens coupling part allowing the lens part to be coupled with the housing by being fixed to in front of the image sensor.

According to another aspect of the present invention, there is provided a method of manufacturing an endoscopic camera module package, the method including: mounting an image sensor and a light source part on a flexible printed circuit board; and coupling a lens part and the flexible printed circuit board into a housing. Here, the housing includes an image sensor supporting frame allowing the image sensor to be supported at a rear of the lens part while the flexible printed circuit board is bent.

The image sensor supporting frame may allow the flexible printed circuit board to be bent by being formed to be spaced apart from the lens part at the rear of the lens part while having a predetermined height in a direction perpendicular to the optical axis.

The flexible printed circuit board may have a light source part mounted thereon in order to provide lighting for photography, and the light source part may be positioned in front of the image sensor.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is an exploded perspective view illustrating an endoscopic camera module package according to an exemplary embodiment of the present invention;

FIG. 2 is a cut-away perspective view illustrating an endoscopic camera module package according to an exemplary embodiment of the present invention;

FIG. 3 is a cross-sectional view taken along A-A′ shown in FIG. 2;

FIG. 4A is a cross-sectional view illustrating an endoscopic camera module package prior to assembly according to an exemplary embodiment of the present invention; and

FIG. 4B is a cross-sectional view illustrating an endoscopic camera module package after assembly according to an exemplary embodiment of the present invention.

### DETAILED DESCRIPTION OF THE PREFERRED EMBODIMENT

Exemplary embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In describing the present invention, if a detailed explanation for a related known function or construction is considered to unnecessarily divert the gist of the present invention, such explanation will be omitted but would be understood by those skilled in the art.

In the drawings, the same reference numerals will be used throughout to designate the same or like elements.

In addition, in a whole disclosure, when one element is referred to as being “connected” to another element, it should be understood that the former can be “directly connected” to the latter, or “indirectly connected” to the latter via an intervening element. Furthermore, “including” an element signifies further including, not excluding, another element if there is no specific reference to the contrary.

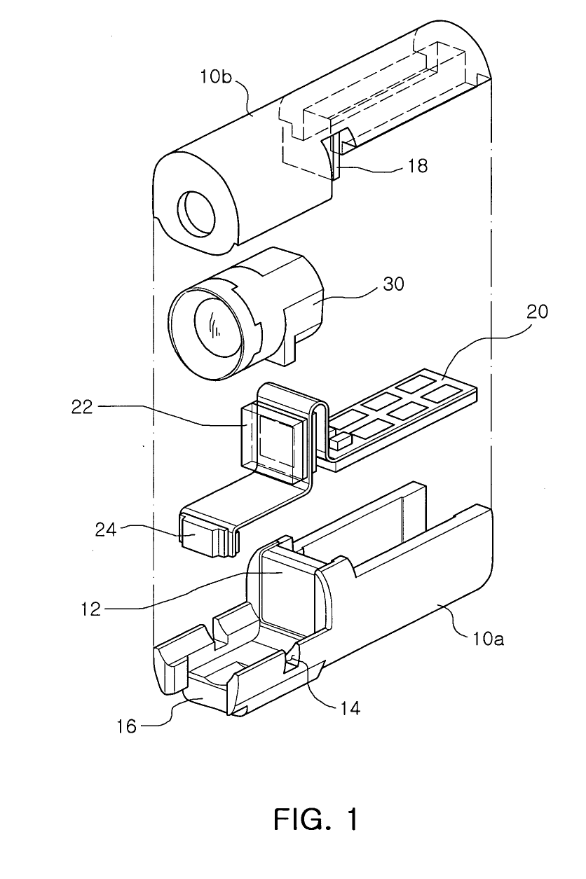

FIG. 1 is an exploded perspective view illustrating an endoscopic camera module package according to an exemplary embodiment of the present invention.

Referring to FIG. 1, an endoscopic camera module package according to this embodiment of the invention may include a lower housing 10a, an upper housing 10b, a flexible printed circuit board (hereinafter, referred to as “FPCB”) 20, an image sensor 22, a light source part 24, and a lens part 30.

The lens part 30 may have an inner space in order to accommodate a lens and have a hole in order that light passes through the accommodated lens along an optical axis.

The image sensor 22 provides an imaging area where light forms an image. The image sensor 22 is disposed at the rear of the lens part 30 in a direction perpendicular to the optical axis such that the light passing through the lens part 30 along the optical axis may form an image.

The light source part 24 provides lighting in order to enable a photograph to be taken in the dark interior of a human body. Here, the light source part 24 may be a chip light emitting diode (LED).

The FPCB 20 may have the image sensor 22 and the light source part 24 mounted thereon. Also, the FPCB 20 may have a variety of passive elements mounted thereon to process signals of the image formed in the image sensor 22.

The upper and lower housings 10b and 10a are coupled with each other in such a manner that the lens part 30, the FPCB 20, the image sensor 22, and the light source part 24 are installed therein, thereby protecting these elements from external impact.

Here, the upper housing 10b may have an opening along the optical axis so that light can be introduced into the lens of the lens part 30.

The lower housing 10a may have a light source coupling part 16 formed by being recessed inwards with a predetermined depth at the front of the lower housing 10a, in order to allow the light source part 24 to be disposed in the direction perpendicular to the optical axis.

The lower and upper housings 10a and 10b have an image sensor supporting frame 12 and a bending frame 18 respectively formed therein, so that the FPCB 20 may be bent when coupled with the housings. Specifically, the image sensor supporting frame 12 may allow the FPCB 20 to be bent by being formed to be spaced apart from the lens part 30 at the rear of the lens part 30 while having a predetermined height in the direction perpendicular to the optical axis.

The lower housing 10a has a lens coupling part 14 with which a coupling member formed on the bottom surface of the lens part 30 is coupled, thereby preventing the lens part 30 from being movable inside the housings.

The light source part 24 may be disposed in substantially the same plane with respect to the front surface of the lens part 30 in the direction perpendicular to the optical axis as shown in FIG. 1. Therefore, the blocking of lighting by the lens part 30 may be avoided, and sufficient lighting is ensured by positioning the light source part 24 closer to a subject for photography.

Also, in the endoscopic camera module package according to this embodiment of the invention, the image sensor 22 and the light source part 24 are mounted on the FPCB 20, and accordingly, there is no need for additional processes and elements conventionally required when a PCB having an image sensor mounted thereon is connected to a separate PCB after a light source part is mounted on that separate PCB.

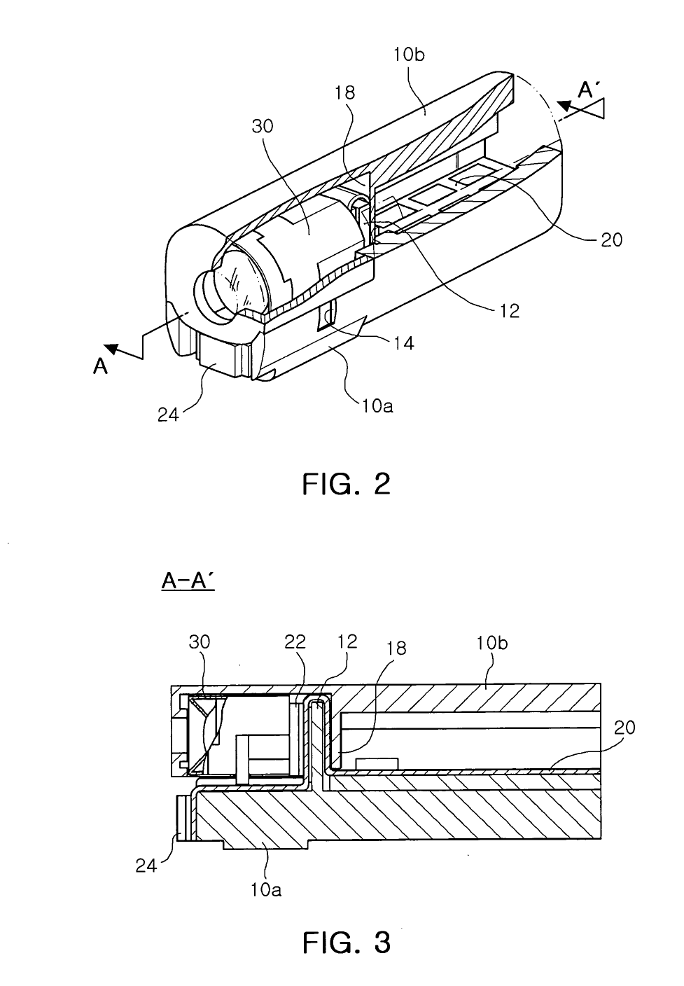

FIG. 2 is a cut-away perspective view illustrating an endoscopic camera module package according to an exemplary embodiment of the present invention.

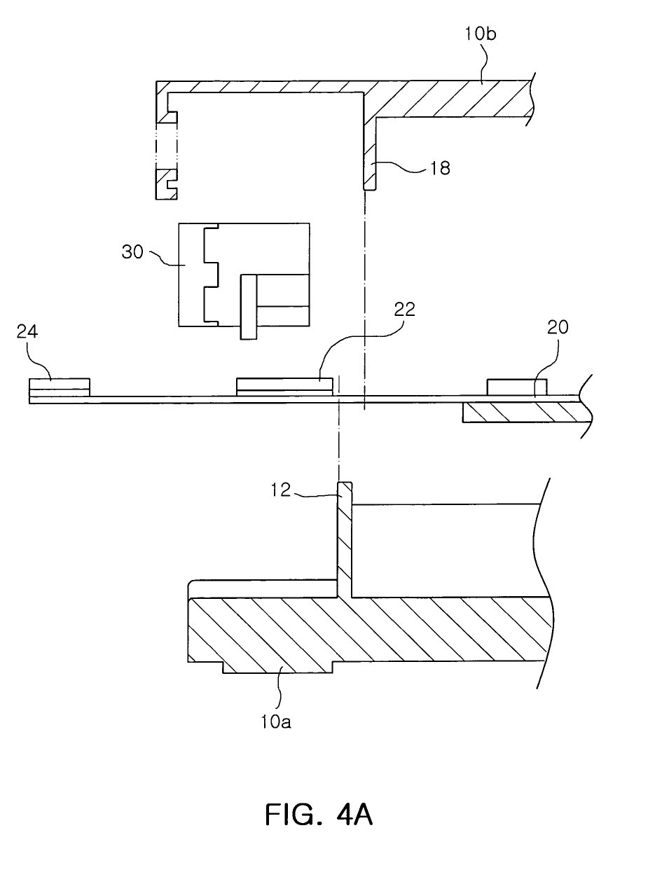

FIG. 3 is a cross-sectional view taken along A-A′ shown in FIG. 2.

In FIGS. 2 and 3, the coupling of the lens part 30, the FPCB 20, the image sensor 22 and the light source part 24 into the upper and lower housings 10b and 10a is illustrated in detail.

The lens part 30 may be fixedly coupled to the lens coupling part 14 formed on the lower housing 10a in such a manner that light introduced through the opening formed in the upper housing 10b is allowed to pass through the lens.

The light source part 24, when coupled, may be disposed in substantially the same plane with respect to the front surface of the lens part 30, whereby the blocking of lighting is minimized and sufficient lighting is ensured.

When the housings and the elements are assembled, the lens part 30 is positioned at the front of the image sensor supporting frame 12 with a predetermined space therebetween, and the bending frame 18 formed in the upper housing 10b is positioned at the rear of the image sensor supporting frame 12 with a predetermined space therebetween.

Therefore, the FPCB 20 is bent by the pressing of the lens part 30 and the bending frame 18 at the both sides of the image sensor supporting frame 12.

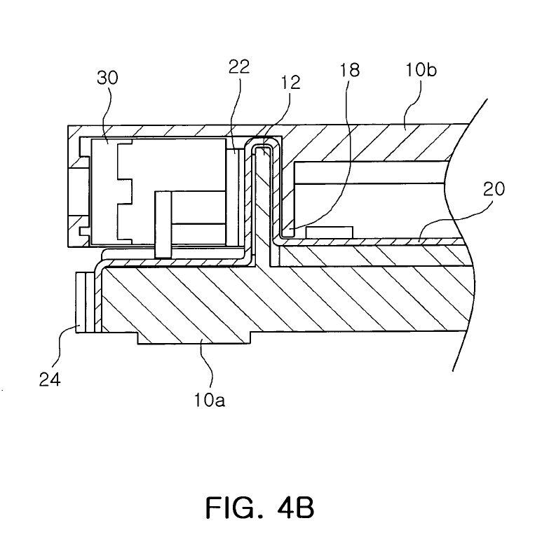

FIGS. 4A and 4B are cross-sectional views illustrating the assembly process of an endoscopic camera module package according to an exemplary embodiment of the present invention.

Referring to FIG. 4A, the upper housing 10b, the FPCB 20 having the lens part 30, the image sensor 22 and the light source part 24 mounted thereon, and the lower housing 10a are sequentially arranged from the upper part.

When the FPCB 20 having the image sensor 22 and the light source part 24 mounted thereon is coupled with the housings, the image sensor 22 and the light source part 24 are arranged to be placed in an appropriate position. Specifically, the image sensor 22 may be arranged in such a manner that its rear edge is positioned slightly in front of the front edge of the image sensor supporting frame 12 formed in the lower housing 10a.

The FPCB 20 maintains a flat shape before being coupled with the housings as shown in FIG. 4A. On this flat top surface of the FPCB 20, the image sensor 22 and the light source part 24 are mounted. Accordingly, both the image sensor 22 and the light source part 24 can be mounted on the FPCB 20 by a single process.

According to the related art, in order to position a light source part in front of the image sensor, after the light source part is mounted on a separate PCB, this PCB is connected to a PCB having an image sensor mounted thereon, and accordingly, additional elements and processes have been required. In the endoscopic camera module package according to this embodiment of the invention, however, those additional elements and processes are not required, whereby the simplified manufacturing process and the reduced manufacturing cost lead to enhanced productivity.

When the upper and lower housings 10b and 10a are coupled with each other in the situation that the elements are arranged as shown in FIG. 4A, the FPCB 20 is coupled into the inside of the housings by being bent as shown in FIG. 4B.

Specifically, the FPCB 20, when coupled, may be bent to conform to the spaces among the lens part 30, the image sensor supporting frame 12, and the bending frame 18.

Therefore, an additional process for bending the FPCB 20 is not required. In other words, when the upper and lower housings 10b and 10a are coupled with each other, the FPCB 20 may be coupled into the housings by being bent.

As set forth above, in the endoscopic camera module package according to exemplary embodiments of the invention, the image sensor and the light source part are mounted on the FPCB, and then the FPCB is coupled into the upper and lower housings by being bent. Also, the light source part is positioned in front of the image sensor. Therefore, the blocking of lighting by the lens part or the like is minimized, and sufficient lighting is ensured by positioning the light source part closer to the subject.

Furthermore, since the FPCB is bent when coupled into the housings, after the image sensor and the light source part are mounted on one FPCB by a single process, additional processes and elements for positioning the light source part in front of the image sensor are not required. Accordingly, productivity is enhanced.

While the present invention has been shown and described in connection with the exemplary embodiments, it will be apparent to those skilled in the art that modifications and variations can be made without departing from the spirit and scope of the invention as defined by the appended claims.

What is claimed is:

1. An endoscopic camera module package comprising:
a lens part including at least one lens arranged along an optical axis;
a flexible printed circuit board having an image sensor mounted thereon, the image sensor allowing light passing through the lens part to form an image; and
a housing including an image sensor supporting frame allowing the image sensor to be supported at a rear of the lens part while the flexible printed circuit board is bent.

2. The endoscopic camera module package of claim 1, wherein the image sensor supporting frame allows the flexible printed circuit board to be bent by being formed to be spaced apart from the lens part at the rear of the lens part while having a predetermined height in a direction perpendicular to the optical axis.

3. The endoscopic camera module package of claim 1, wherein the flexible printed circuit board has a light source part mounted thereon in order to provide lighting for photography, the light source part being positioned in front of the image sensor.

4. The endoscopic camera module package of claim 3, wherein the light source part is a chip light emitting diode (LED).

5. The endoscopic camera module package of claim 3, wherein the housing includes a light source coupling part allowing the light source part to be coupled with the housing in a direction perpendicular to the optical axis.

6. The endoscopic camera module package of claim 1, wherein the housing includes a lens coupling part allowing the lens part to be coupled with the housing by being fixed to in front of the image sensor.

7. A method of manufacturing an endoscopic camera module package, the method comprising:
mounting an image sensor and a light source part on a flexible printed circuit board; and
coupling a lens part and the flexible printed circuit board into a housing,
wherein the housing includes an image sensor supporting frame allowing the image sensor to be supported at a rear of the lens part while the flexible printed circuit board is bent.

8. The method of claim 7, wherein the image sensor supporting frame allows the flexible printed circuit board to be bent by being formed to be spaced apart from the lens part at the rear of the lens part while having a predetermined height in a direction perpendicular to the optical axis.

9. The method of claim 7, wherein the flexible printed circuit board has a light source part mounted thereon in order to provide lighting for photography, the light source part being positioned in front of the image sensor.
